(19) United States
(12) Patent Application Publication
(10) Pub. No.: US 2007/0167438 A1
(43) Pub. Date: Jul. 19, 2007

(54) Treatment of substance abuse

(75) Inventor: Sharon Rosenzweig-Lipson, East Brunswick, NJ (US)

Correspondence Address: CHOATE, HALL & STEWART LLP/WYETH, PATENT GROUP, TWO INTERNATIONAL PLACE, BOSTON, MA, 02110, US

(73) Assignee: Wyeth, Madison, NJ (US)

(21) Appl. No.: 11/653,027

(22) Filed: Jan. 12, 2007

Related U.S. Application Data
(60) Provisional application No. 60/759,148, filed on Jan. 13, 2006.

(51) Int. Cl. A61K 31/551

(52) U.S. Cl. 514/220; 514/291

(57) ABSTRACT

The present invention provides methods and compositions for use in the treatment, prevention, and/or alleviation of drug abuse and/or its symptoms. In particular, the invention demonstrates that compositions comprising compounds of formula I are useful in such treatment, prevention, and/or alleviation: 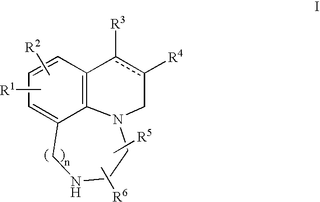
or a pharmaceutically acceptable salt thereof, wherein each of n, R¹, R², R³, R⁴, R⁵, and R⁶ are as defined herein.

### CROSS-REFERENCE TO RELATED APPLICATIONS

The present application claims priority to U.S. provisional patent application Ser. No. 60/759,148, filed, Jan. 13, 2006, the entirety of which is hereby incorporated herein by reference.

### FIELD OF THE INVENTION

The present invention relates to the use of compounds in the treatment of substance abuse and/or its symptoms.

### BACKGROUND OF THE INVENTION

According to the National Survey on Drug Use and Health authored by the Substance Abuse and Mental Health Services Administration of the United States Department of Health and Human Services, an estimated 21.6 million Americans (9.1 percent of the total population aged 12 or older) were classified with substance dependence or abuse in 2003. Of these, 3.1 million were classified with dependence on or abuse of both alcohol and illicit drugs, 3.8 million were dependent on or abused illicit drugs but not alcohol, and 14.8 million were dependent on or abused alcohol but not illicit drugs.

Agents that are abused include those used recreationally to alter mood, thought, or feeling (e.g., cigarettes, alcohol, etc.), those that are prescribed or otherwise administered for therapeutic benefit but upon which dependency develops (e.g., pain relievers, such asfor example, Vicodin®, Lortab®, Lorcet®, Percocet®, Percodan ®, Tylox®, Hydrocodone, OxyContin®, methadone, Tramadol, etc, tranquilizers, stimulants, or sedatives), and those that are obtained illegally for the purpose of achieving a particular physiological effect or “high” (e.g., marijuana, heroine, cocaine, ecstasy, LSD, PCP, methamphetamine, etc.).

Many people who would like to quit use of such abused agents cannot because they are addicted to one or more dependence-inducing components (e.g., opioids, nicotine, etc.). Moreover, treatment for substance abuse often involves transfer of dependence to an alternative, but also dependence-inducing agent. Even successful treatment often involves significant and unpleasant withdrawal symptoms.

There remains a need for improved therapies for the treatment of substance abuse.

### SUMMARY OF THE INVENTION

The present invention provides methods and compositions for the treatment of substance abuse and/or its symptoms, including withdrawal. In particular, the invention encompasses the finding that compounds of formula I are useful in the treatment of substance abuse and/or its symptoms: 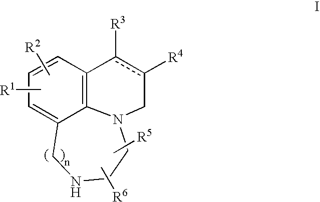
or a pharmaceutically acceptable salt thereof, wherein:
[symbol] designates a single or double bond;
n is 1 or2;
R¹ and R² are each independently halogen, —CN, —R, —OR, —C₁₋₆ perfluoroalkyl, —OC₁₋₆ perfluoroalkyl, or phenyl optionally substituted with one to five groups independently selected from halogen, —R, —OR, —C₁₋₆ perfluoroalkyl, or —OC₁₋₆ perfluoroalkyl;
each R is independently hydrogen or a C₁₋₆ alkyl group;
R³ and R⁴ are taken together, with the carbon atoms to which they are bound, to form a saturated or unsaturated 4-8 membered carbocyclic ring, wherein said ring is optionally substituted with 1-3 groups independently selected from halogen, —R, or —OR; and
R⁵ and R⁶ are each independently —R.

For example, the invention provides methods that involve administering to an individual in need thereof a therapeutically effective amount of a compound of formula I, or a pharmaceutically acceptable salt, prodrug, or metabolite thereof. The present invention also provides compositions comprising a compound of formula I or a pharmaceutically acceptable salt thereof, and one or more pharmaceutically acceptable carriers, excipients, or diluents, formulated for the treatment of substance abuse.

### DESCRIPTION OF THE DRAWING

FIG. 1 shows inhibition by a compound of formula I of hyperactivity produced by cocaine.

### DETAILED DESCRIPTION OF CERTAIN EMBODIMENTS OF THE INVENTION

1. Compounds and Definitions:

As noted above, the present invention provides methods and compositions for the treatment of substance abuse and/or its symptoms, including withdrawal.

In general, compounds that are useful in accordance with the present invention have the structure presented in formula I: 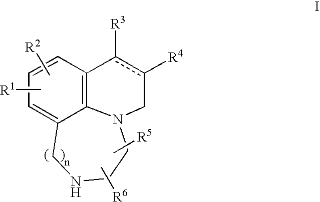
or a pharmaceutically acceptable salt thereof, wherein:
[symbol] designates a single or double bond;
n is 1 or 2;
R¹ and R² are each independently halogen, —CN, —R, —OR, —C_{1-b 6} perfluoroalkyl, —OC₁₋₆ perfluoroalkyl, or phenyl optionally substituted with one to five groups independently selected from halogen, —R, —OR, —C₁₋₆ perfluoroalkyl, or —OC₁₋₆ perfluoroalkyl;
each R is independently hydrogen or a C₁₋₆ alkyl group;
R³ and R⁴ are taken together, with the carbon atoms to which they are bound, to form a saturated or unsaturated 4-8 membered carbocyclic ring, wherein said ring is optionally substituted with 1-3 groups independently selected from halogen, —R, or —OR; and
R⁵ and R⁶ are each independently —R.

As used herein, the term “alkyl”, includes, but is not limited to, straight and branched chains such as methyl, ethyl, n-propyl, isopropyl, n-butyl, isobutyl, sec-butyl, or t-butyl. In certain embodiments, the term “alkyl” refers to straight and branched chains having from 1 to 3 carbon atoms.

The terms “halogen” or “halo,” as used herein, refer to chlorine, bromine, fluorine or iodine.

The term “perfluoroalkyl,” as used herein refers to an alkyl group, as defined herein, wherein every hydrogen atom on said alkyl group is replaced by a fluorine atom. Such perfluoroalkyl groups include —CF₃.

The terms “effective amount” and “therapeutically effective amount,” as used herein, refer to the amount of a composition of the present invention that, when administered to a patient, is effective to at least partially treat a condition from which the patient is suffering from.

The term “pharmaceutically acceptable salts” or “pharmaceutically acceptable salt” refers to salts derived from treating a compound of formula I with an organic or inorganic acid such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, or similarly known acceptable acids. In certain embodiments, the present invention provides the hydrochloride salt of a compound of formula I.

The term “patient,” as used herein, refers to a mammal. In certain embodiments, the term “patient” refers to a human.

The terms “administer,” “administering,” or “administration,” as used herein, refer to either directly administering a compound or composition to a patient, or administering a prodrug derivative or analog of the compound to the patient, which will form an equivalent amount of the active compound or substance within the patient's body.

The terms “treat” or “treating,” as used herein, refers to partially or completely alleviating, inhibiting, preventing, ameliorating and/or relieving the condition, or one or more symptoms thereof. Those of ordinary skill in the art will appreciate that substance abuse often involves symptoms of physical and/or psychological dependence. Also, when the substance of abuse is withdrawn from a dependent individual, the individual often develops certain symptoms including sleep and mood disturbance and intense craving of the substance of abuse, known as “withdrawal”. The present invention encompasses treatment of substance abuse itself, dependence, and also of withdrawal.

“Withdrawal” refers to a collection of symptoms that arise when administration of a relevant substance is reduced, delayed, or stopped. The substance-specific symptoms of withdrawal can cause clinically significant distress or impairment in social, occupational or other important areas of functioning. These symptoms are not due to a general medical condition and are not better accounted for by another mental disorder. Withdrawal usually, but not necessarily, is associated with substance dependence.

Withdrawal symptoms can arise upon reduction of any of a variety of substances. For example, the discontinued use of tobacco products, all of which contain nicotine, typically results in the onset of nicotine withdrawal conditions. Individuals often suffer the symptoms of nicotine withdrawal as a consequence of the discontinued use of tobacco in any form, including, but not limited to smoking of cigarette, cigar, or pipe tobacco, or the oral or intranasal ingestion of tobacco or chewing tobacco. Such oral or intranasal tobacco includes, but is not limited to snuff and chewing tobacco. The cessation of nicotine use or reduction in the amount of nicotine use, is often followed within 24 hours by symptoms including dysphoric, depressed mood; light-headedness; insomnia; irritability, frustration or anger; anxiety; nervous tremor; difficulty concentrating; restlessness; decreased heart rate; increased appetite or weight gain; and the craving for tobacco or nicotine. These symptoms often cause clinically significant distress or impairment in social, occupational, or other important areas of functioning. The present invention is most preferably used to alleviate one or more symptoms attributed to nicotine withdrawal when such symptoms are not due to a general medical condition and are not better accounted for by another medical disorder. The present method is also helpful to those who have replaced, or partially replaced, their use of tobacco with the use of nicotine replacement therapy. Thus, such patients can be assisted to reduce and even eliminate entirely their dependence on nicotine in all forms.

The discontinuation or reduction in administration of an opioid, typically self-administration, through injection or orally, through smoking or intranasal ingestion, often results in the presence of a characteristic opioid withdrawal condition. This withdrawal condition can also be precipitated by administration of an opioid antagonist such as naloxone or naltrexone after opioid use. Opioid withdrawal is characterized by symptoms that are generally opposite to the opioid agonist effects. These withdrawal symptoms may include anxiety; restlessness; muscle aches, often in the back and legs; craving for opioids; irritability and increased sensitivity to pain; dysphoric mood; nausea or vomiting; lacrimation; rhinorrhoea; papillary dilation; piloerection; sweating; diarrhea; yawning; fever; and insomnia. When dependence is on short-acting opioids, such as heroin, withdrawal symptoms usually occur within 6-24 hours after the last dose, while with longer-acting opioids, such as methadone, symptoms may take 2-4 days to emerge. These symptoms often cause clinically significant distress or impairment in social, occupational or other important areas of functioning. The present invention is most preferably used to alleviate one or more symptoms attributed to opioid withdrawal when such symptoms are not due to a general medical condition and are not better accounted for by another medical disorder.

The discontinued or reduction in use of ethanol (ethanol containing beverages) results in the onset of ethanol withdrawal conditions. Ethanol withdrawal conditions are characterized by symptoms that begin when blood concentrations of ethanol decline sharply, within 4 to 12 hours after ethanol use has been stopped or reduced. These ethanol withdrawal symptoms include craving for ethanol; autonomic hyperactivity (such as sweating or pulse rate greater than 100); hand tremor; insomnia; nausea; vomiting; transient visual, tactile, or auditory hallucinations or illusions; psychomotor agitation; anxiety; and grand mal seizures. These symptoms often cause clinically significant distress or impairment in social, occupational, or other important areas of functioning. The present invention is most preferably used to alleviate one or more symptoms attributed to ethanol withdrawal when such symptoms are not due to a general medical condition and are not better accounted for by another medical disorder.

The terms “suffer” or “suffering” as used herein refers to one or more conditions that a patient has been diagnosed with, or is suspected to have.

The term “substance abuse”, as used herein, may be defined with reference to criteria set forth in the Diagnostic and Statistical Manual of Mental Disorders, 4^{th} Ed. (1994) (“DSM-IV”), which was prepared by the Task Force on Nomenclature and Statistics of the American Psychiatric Association. A feature of substance abuse is a maladaptive pattern of substance use manifested by recurrent and significant adverse consequences related to the repeated use of substances. As recited in the DSM-IV, substance abuse is defined as maladaptive pattern of substance abuse leading to clinically significant impairment or distress, as manifested by one (or more) of the following, occurring within a 12-month period: (1) recurrent substance use resulting in a failure to fulfill major role obligations at work, school, or home; (2) recurrent substance use in situations in which it is physically hazardous; (3) recurrent substance-related legal problems; and (4) continued substance use despite having persistent or recurrent social or interpersonal problems caused or exacerbated by the effects of the substance. In addition, the DMS-IV requires that the symptoms of substance abuse do not meet the criteria for substance dependence.

The term “substance dependence”, as used herein, may be defined with reference to criteria set form in the Diagnostic and Statistical Manual of Mental Disorders, 4^{th} Ed. (1994) (“DSM-IV”), which was prepared by the Task Force on Nomenclature and Statistics of the American Psychiatric Association. The criteria for substance dependence set forth in DSM-IV is a pattern of substance use, leading to clinically significant impairment or distress as manifested by at least three selected from the following group, occurring at any time within the same twelve month period: (1) tolerance as defined by either (a) a need for substantially increased amounts of the substance to achieve the desired effect; or (b) substantially diminished effect with continued use of the same amount of the substance; (2) withdrawal, as demonstrated by either (a) the characteristic withdrawal syndrome for the specific substance; or (b) the same, or a closely related substance is taken to relieve or avoid withdrawal symptoms; (3) the substance is often taken in larger amounts or over a longer period then was intended; (4) there is a persistent desire or unsuccessful efforts to cut down or control substance use; (5) a great deal of time is spent in activities to obtain the substance, use the substance, or recover from its effects; (6) important social, occupational or recreational activities are given up or reduced because of substance use; and (7) the substance use is continued despite knowledge of having a persistent or recurrent physical or psychological problem that is likely to have been caused or exacerbated by the substance. Substance dependence can be with physiological dependence; that is evidence of tolerance or withdrawal is present, or without physiological dependence, where no evidence of tolerance or withdrawal is present. Four of the conditions set forth in DSM-IV include remission. These types of remission are based on the interval of time that has elapsed since the cessation of dependencies and whether there is continued presence of one or more of the symptoms included in the criteria for dependencies.

The qualifier “early full remission” is used when for at least one month, but for less than twelve months, no symptom of dependence has been met.

The qualifier “early partial remission” is used when for at least one month but less than 12 months, one or more symptoms for dependence has been met, but the full criteria for dependence has not been met.

The term “sustained full remission” is used when none of the symptoms of dependence have been met at any time during a period of twelve months or longer.

The term “sustained partial remission” is used if the symptoms for dependence have not been met for a period of twelve months or longer, however, one or more symptom for dependence has been met.

The qualifier “on agonist therapy” is used if the subject is on a prescribed agonist medication and no symptom for dependence has been met for that class of medication for at least the past month. It also applies to those being treated for dependence using a partial agonist.

The term “in a controlled environment” is used if the subject is in an environment where access to substances of abuse are restricted and no symptom for dependence has been met for at least the past month.

The term “cessation and withdrawal” shall include, but is not limited to, the following conditions characterized in the DSM-IV: Nicotine Withdrawal; Nicotine-Related Disorder Not otherwise Specified; Nicotine Dependence, with physiological dependence; Nicotine Dependence, without physiological dependence; Nicotine Dependence, Early Full Remission; Nicotine Dependence, Early Partial Remission; Nicotine Dependence, Sustained Full Remission; Nicotine Dependence, Sustained Partial Remission; Nicotine Dependence, On Agonist Therapy; Opioid Withdrawal; Opioid-Related Disorder Not Otherwise Specified; Opioid Dependence, with physiological dependence; Opioid Dependence, without physiological dependence; Opioid Dependence, Early Full Remission; Opioid Dependence, Early Partial Remission; Opioid Dependence, Sustained Full Remission; Opioid Dependence, Sustained Partial Remission; Opioid Dependence On Agonist Therapy; and Opioid Dependence in a controlled environment; Ethanol Withdrawal; Ethanol Dependence with Physiological Dependence; Ethanol Withdrawal, without Physiological Dependence; Ethanol Withdrawal, Early Full Remission; Ethanol Withdrawal, Early Partial Remission; Ethanol Withdrawal, Sustained Full Remission; Ethanol Withdrawal, Sustained Partial Remission; Ethanol Withdrawal, on Agonist Therapy; and Ethanol Withdrawal, In a Controlled Environment.

2. Description of Exemplary Compounds:

In certain embodiments, the present invention provides a compound of formula I: 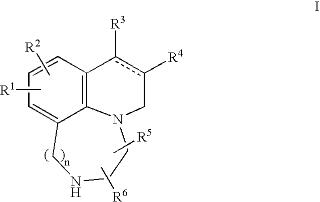
or a pharmaceutically acceptable salt thereof for use in treating one or more symptoms of substance abuse and/or its symptoms, wherein:
[symbol] designates a single or double bond;
n is 1 or 2;
R¹ and R² are each independently halogen, —CN, —R, —OR, —C₁₋₆ perfluoroalkyl, —OC₁₋₆ perfluoroalkyl, or phenyl optionally substituted with one to five groups independently selected from halogen, —R, —OR, —C₁₋₆ perfluoroalkyl, or —OC₁₋₆ perfluoroalkyl;
each R is independently hydrogen or a C₁₋₆ alkyl group;
R³ and R⁴ are taken together, with the carbon atoms to which they are bound, to form a saturated or unsaturated 4-8 membered carbocyclic ring, wherein said ring is optionally substituted with 1-3 groups independently selected from halogen, —R, or —OR; and
R⁵ and R⁶ are each independently —R.

The compounds of formula I, as defined above or in classes and subclasses as described herein, have affinity for and agonist or partial agonist activity at the 2C subtype of brain serotonin receptors.

In certain embodiments, [symbol] designates a single bond. In other embodiments, [symbol] designates a double bond.

In certain embodiments, the R¹ group of formula I is R, OR, halogen, cyano, or —C₁₋₃ perfluoroalkyl. In other embodiments, the R¹ group of formula I is hydrogen, halogen, cyano, —OR wherein R is C₁₋₃ alkyl, or trifluoromethyl. According to another embodiment, the R¹ group of formula I is hydrogen.

In certain embodiments, the R² group of formula I is R, OR, halogen, cyano, or —C₁₋₃ perfluoroalkyl. In other embodiments, the R² group of formula I is hydrogen, halogen, cyano, —OR wherein R is hydrogen, C₁₋₃ alkyl, or trifluoromethyl. According to another embodiment, the R² group of formula I is hydrogen.

According to one aspect of the present invention, at least one of R¹ and R² groups of formula I is —OH. According to another aspect of the present invention, both of the R¹ and R² groups of formula I are —OH.

According to another embodiment, each of the R¹ and R² groups of formula I is hydrogen. According to yet another embodiment, each of the R⁵ and R⁶ groups of formula I is hydrogen.

As defined generally above, the R³ and R⁴ groups of formula I are taken together to form a saturated or unsaturated 4-8 membered carbocyclic ring, wherein said ring is optionally substituted with 1-3 groups independently selected from halogen, —R, or OR. According to one embodiment, the R³ and R⁴ groups of formula I are taken together to form a saturated or unsaturated 5-8 membered carbocyclic ring, wherein said ring is optionally substituted with 1-3 groups independently selected from halogen, —R, or OR. In certain embodiments, the R³ and R⁴ groups of formula I are taken together to form a saturated or unsaturated 5-6 membered carbocyclic ring, wherein said ring is optionally substituted with 1-3 groups independently selected from halogen, —R, or OR.

As defined generally above, n is 1 or 2. Accordingly, the present invention provides methods and compositions using a compound of formulae I-a and I-b: 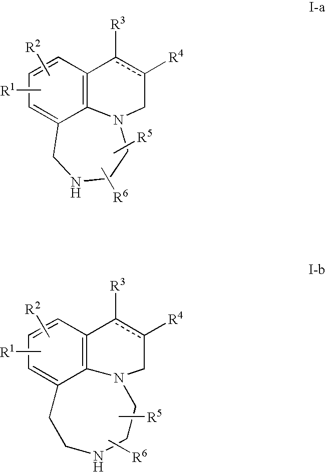
or a pharmaceutically acceptable salt thereof, wherein each of R¹, R², R³, R⁴, R⁵, and R⁶ is as defined above for compounds of formula I and described in classes and subclasses above and herein.

In other embodiments, n is 1 and the R³ and R⁴ groups of formula I are taken together to form a saturated 5-membered carbocyclic ring and said compound is of formula II: 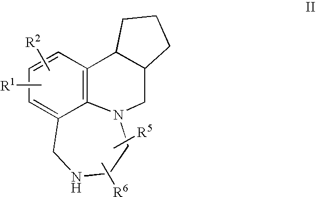
or a pharmaceutically acceptable salt thereof, wherein each of R¹, R², R⁵, and R⁶ is as defined above for compounds of formula I and described in classes and subclasses above and herein.

Compounds of the present invention contain asymmetric carbon atoms and thus give rise to stereoisomers, including enantiomers and diastereomers. Accordingly, it is contemplated that the present invention relates to all of these stereoisomers, as well as to mixtures of the stereoisomers. Throughout this application, the name of the product of this invention, where the absolute configuration of an asymmetric center is not indicated, is intended to embrace the individual stereoisomers as well as mixtures of stereoisomers.

According to another aspect, the present invention provides methods and compositions using a compound of either of formulae I-c or I-d: 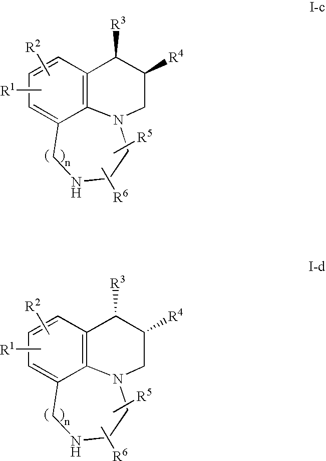
or a pharmaceutically acceptable salt thereof, wherein each of n, R¹, R², R³, R⁴, R⁵, and R⁶ is as defined above for compounds of formula I and described in classes and subclasses above and herein.

In certain embodiments, the present invention provides methods and compositions using a compound of formula III: 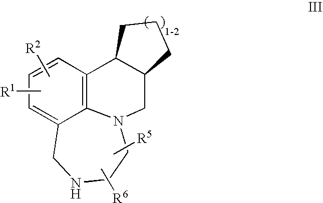
or a pharmaceutically acceptable salt thereof, wherein each R¹, R², R⁵, and R ⁶ are as defined above for compounds of formula I and in classes and subclasses as described above and herein.

Where an enantiomer is preferred, it may, in some embodiments be provided substantially free of the corresponding enantiomer. Thus, an enantiomer substantially free of the corresponding enantiomer refers to a compound which is isolated or separated via separation techniques or prepared free of the corresponding enantiomer. “Substantially free,” as used herein, means that the compound is made up of a significantly greater proportion of one enantiomer. In certain embodiments the compound is made up of at least about 90% by weight of a preferred enantiomer. In other embodiments of the invention, the compound is made up of at least about 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

Exemplary compounds useful for the methods of the present invention are set forth in Table 1, below.

Table 1. Exemplary Compounds of Formula I

4,5,6,7,9a,10,11,12,13,13a-decahydro-9H-[1,4]diazepino[6,7,1-de]phenanthridine;
2-bromo-4,5,6,7,9,9a,10,11,12,12a-decahydrocyclopenta[c][1,4]diazepino[6,7,1-ij]quinoline;
2-bromo-4,5,6,7,9,9a,10,11,12,13,14,14a-dodecahydrocyclohepta[c][1,4]diazepino[6,7,1-ij]quinoline;
2-chloro-4,5,6,7,9,9a,10,11,12,12a-decahydrocyclopenta[c][1,4]diazepino[6,7,1-ij]quinoline;
2-chloro-4,5,6,7,9,9a,10,11,12,13,14,14a-dodecahydrocyclohepta[c][1,4]diazepino[6,7,1-ij]quinoline;
2-phenyl-4,5,6,7,9,9a,10,11,12,12a-decahydrocyclopenta[c][1,4]diazepino[6,7,1-ij]quinoline;
2-methoxy-4,5,6,7,9,9a,10,11,12,12a-decahydrocyclopenta[c][1,4]diazepino[6,7,1-ij]quinoline;
1-fluoro-4,5,6,7,9,9a,10,11,12,12a-decahydrocyclopenta[c][1,4]diazepino[6,7,1-ij]quinoline;
1-fluoro-4,5,6,7,9,9a,10,11,12,13,14,14a-dodecahydrocyclohepta[c][1,4]diazepino[6,7,1-ij]quinoline;
1-(trifluoromethyl)-4,5,6,7,9,9a,10,11,12,12a-decahydrocyclopenta[c][1,4]diazepino[6,7,1-ij]quinoline;
1-fluoro-2-methoxy-4,5,6,7,9,9a,10,11,12,12a-decahydrocyclopenta[c][1,4]diazepino[6,7,1-ij]quinoline;
1-fluoro-2-methoxy-4,5,6,7,9,9a,10,11,12,13,14,14a-dodecahydrocyclohepta[c][1,4]diazepino[6,7,1-ij]quinoline;
4,5,6,7,9,9a10,11,12,12a-decahydrocyclopenta[c][1,4]diazepino[6,7,1-ij]quinoline;
4,5,6,7,9,9a,10,11,12,13,14,14a-dodecahydrocyclohepta[c][1,4]diazepino[6,7,1-ij]quinoline;
4,5,6,7,9,9a,10,11,12,12a-decahydrocyclopenta[c][1,4]diazepino[6,7,1-ij]quinoline;
(9aR,12aS)-4,5,6,7,9,9a,10,11,12,12a-decahydrocyclopenta[c][1,4]diazepino[6,7,1-ij]quinoline;
(9aS,12aR)-4,5,6,7,9,9a,10,11,12,12a-decahydrocyclopenta[c][1,4]diazepino[6,7,1-ij]quinoline;
(9aR,14aS)-4,5,6,7,9,9a,10,11,12,13,14,14a-dodecahydrocyclohepta[c][1,4]diazepino[6,7,1-ij]quinoline; or
(9aS,14aR)-4,5,6,7,9,9a,10,11,12,13,14,14a-dodecahydrocyclohepta[c][1,4]diazepino[6,7,1-ij]quinoline;
   or a pharmaceutically acceptable salt thereof, including the hydrochloride salt of each of the above compounds.
   3. General Methods of Providing the Present Compounds:

Compounds of formula I for use in accordance with the present invention may be obtained or produced according to any available means including methods described in detail in U.S. patent application Ser. No. 10/422,524, filed Apr. 24, 2003 and Ser. No 11/267,448 filed Nov. 4, 2005, and in U.S. provisional patent application Ser. Nos. 60/702,509, filed Jul. 26, 2005 and 60/727,606 filed Oct. 17, 2005, the entirety of each of which is hereby incorporated herein by reference.

4. Uses, Formulation and Administration

According to the present invention, compounds of formula I may be used to treat, prevent, or alleviate dependence, withdrawal, or symptoms thereof for any of a variety of substances including, for example, recreational substances (e.g., alcohol, tobacco), pharmacologic agents (e.g., pain relievers [for example, Vicodin®, Lortab®, Lorcet®, Percocet®, Percodan®, Tylox®, Hydrocodone, OxyContin®, methadone, Tramadol, etc], tranquilizers, stimulants, or sedatives), and illicit drugs (e.g., marijuana, heroine, cocaine, ecstasy, LSD, PCP, methamphetamine, etc.).

In evaluating substance abuse in accordance with the present invention, reference may be made, for example, to the National Survey on Drug Use and Health (NSDUH), which obtains information on nine different categories of illicit drug use: marijuana, cocaine, heroin, hallucinogens, inhalants, and nonmedical use of prescription-type pain relievers, tranquilizers, stimulants, and sedatives. In these categories, hashish is included with marijuana, and crack is considered a form of cocaine. Several drugs are grouped under the hallucinogens category, including LSD, PCP, peyote, mescaline, mushrooms, and “Ecstasy” (MDMA). Inhalants include a variety of substances, such as amyl nitrite, cleaning fluids, gasoline, paint, and glue. The four categories of prescription-type drugs (pain relievers, tranquilizers, stimulants, and sedatives) cover numerous drugs available through prescriptions and sometimes illegally “on the street.” Methamphetamine is considered a type of stimulant. Respondents are asked to report only uses of drugs that were not prescribed for them or drugs they took only for the experience or feeling they caused. Over-the-counter drugs and legitimate uses of prescription drugs are not included. NSDUH reports combine the four prescription-type drug groups into a category referred to as “any psychotherapeutics.”

The NSDUH categorizes alcohol abuse through use of questions about the frequency of the consumption of alcoholic beverages, such as beer, wine, whiskey, brandy, and mixed drinks. An extensive list of examples of the kinds of beverages covered is given to respondents prior to the question administration. A “drink” is defined as a can or bottle of beer, a glass of wine or a wine cooler, a shot of liquor, or a mixed drink with liquor in it. Times when the respondent only had a sip or two from a drink are not considered as consumption. For this report, estimates for the prevalence of alcohol use are reported primarily at three levels defined for both males and females and for all ages as follows:
Current use—At least one drink in the past 30 days (includes binge and heavy use).
Binge use—Five or more drinks on the same occasion at least once in the past 30 days (includes heavy use).
Heavy use—Five or more drinks on the same occasion on at least 5 different days in the past 30 days

The NSDUH also characterizes the use of tobacco products, including cigarettes, chewing tobacco, snuff, cigars, and pipe tobacco. For analytic purposes, data for chewing tobacco and snuff are combined as “smokeless tobacco.” Cigarette use is defined as smoking “part or all of a cigarette.” Questions to determine nicotine dependence among current cigarette smokers also are included in NSDUH. Nicotine dependence is based on criteria from the Nicotine Dependence Syndrome Scale (NDSS) or the Fagerstrom Test of Nicotine Dependence (FTND).

The present invention provides methods of treating, each of the conditions listed above in a patient, preferably in a human, the methods including administering a therapeutically effective amount of at least one compound of formula I or a pharmaceutically acceptable salt thereof to an individual engaged in or susceptible to substance dependence or abuse, and/or to an individual suffering from substance withdrawal.

In certain embodiments, compounds of the present invention are useful for treating alcoholism (e.g., alcohol abuse, addiction and/or dependence including treatment for abstinence, craving reduction and relapse prevention of alcohol intake) and/or tobacco abuse (e.g., smoking addiction, cessation and/or dependence including treatment for craving reduction and relapse prevention of tobacco smoking).

5. Pharmaceutical Compositions

The present invention also relates to compositions comprising at least one compound of formula I, or a pharmaceutically acceptable salt thereof, and one or more pharmaceutically acceptable carriers, excipients, or diluents, formulated for administration to treat, prevent, or alleviate substance dependence or abuse, and/or their symptoms. Such pharmaceutical formulations may be prepared in accordance with acceptable pharmaceutical procedures, such as, for example, those described in Remingtons Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers are those carriers that are compatible with the other ingredients in the formulation and are biologically acceptable.

In certain embodiments, compounds of formula I are the only pharmacologically active ingredients in the composition; in other embodiments one or more other agents is included, for example to treat the same or different symptoms of substance dependence or abuse.

In some embodiments of the invention, therapy utilizing compounds of formula I is administered concomitantly with, in connection with, and/or subsequent to an educational and/or behavioral modification program to enhance continued abstinence from substance dependence or abuse. The method of the present invention may be particularly useful in treating symptoms of withdrawal often observed in rehabilitation or other treatment programs. Therefore, the programs can be more effective by focusing on educational and behavioral modification goals, further reducing the incidence of program non-completion.

The compounds of formula I can be administered orally or parenterally, neat, or in combination with conventional pharmaceutical carriers. Applicable solid carriers can include one or more substances that can also act as flavoring agents, lubricants, solubilizers, suspending agents, fillers, glidants, compression aids, binders, tablet-disintegrating agents, or encapsulating materials. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers can be used in preparing solutions, suspensions, emulsions, syrups and elixirs. The active ingredient can be dissolved or suspended in a pharmaceutically acceptable liquid carrier such as water, an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration, the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions that are sterile solutions or suspensions can be administered by, for example, intramuscular, intraperitoneal or subcutaneous injection. Sterile solutions can also be administered intravenously. Compositions for oral administration can be in either liquid or solid form.

The compounds of formula I can be administered rectally or vaginally in the form of a conventional suppository. For administration by intranasal or intrabronchial inhalation or insufflation, the compounds of formula I can be formulated into an aqueous or partially aqueous solution, which can then be utilized in the form of an aerosol. The compounds of formula I can also be administered transdermally through the use of a transdermal patch containing the active compound and a carrier that is inert to the active compound, is non-toxic to the skin, and allows delivery of the agent for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active ingredient can also be suitable. A variety of occlusive devices can be used to release the active ingredient into the blood stream such as a semipermeable membrane covering a reservoir containing the active ingredient with or without a carrier, or a matrix containing the active ingredient. Other occlusive devices are known in the literature.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form.

The amount of compound of formula I provided to a patient will vary depending upon what is being administered, the purpose of the administration, such as prophylaxis or therapy, the state of the patient, the manner of administration, and the like. In therapeutic applications, compounds of Formula I are provided to a patient suffering from a condition in an amount sufficient to treat or at least partially treat the symptoms of the condition and its complications. An amount adequate to accomplish this is a “therapeutically effective amount” as described previously herein. The dosage to be used in the treatment of a specific case must be subjectively determined by the attending physician. The variables involved include the specific condition and the size, age, and response pattern of the patient. The treatment of substance dependence or abuse follows the same method of subjective drug administration under the guidance of the attending physician. Generally, a starting dose may about 0.5 mg per day with gradual increase in the daily dose to about 500 mg per day, to provide the desired dosage level in the patient. A suitable dose of a compound of formula I for use in the practice of the present invention is expected to be within the range of about 0.5 to about 500 mg, or about 1 mg to 500 mg.

In certain embodiments, the present invention relates to use of prodrugs of compounds of formula I. The term “prodrug,” as used herein, means a compound that is convertible in vivo by metabolic means (e.g. by hydrolysis) to a compound of formula I. Various forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991), Bundgaard, et al., Journal of Drug Delivery Reviews, 8:1-38(1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is hereby incorporated by reference in its entirety.

According to another embodiment, a compound of the present invention is administered in combination with one or more agents useful for treating substance abuse. In certain embodiments, a compound of the present invention is administered in combination with one or more agents to treat tobacco abuse. Such agents include nicotine receptor partial agonists bupropion hypochloride (Zyban™) and nicotine replacement therapies.

According to yet another embodiment, a compound of the present invention is administered in combination with one or more agents to treat alcoholism, such as opioid antagonists (e.g., naltrexone, N-methylnaltrexone, ReVia™), nalmefene, disulfiram (Antabuse™), and acamprosate (Campral™).

In certain embodiments, a compound is administered in combination with one or more agents for reducing alcohol withdrawal symptoms such as benzodiazepines, beta-blockers, clonidine, carbamazepine, pregabalin, and gabapentin (Neurontin™). In other embodiments of the invention, therapy utilizing compounds of the present invention is administered concomitantly with, in connection with, and/or subsequent to an educational and/or behavioral modification program to enhance continued abstinence from substance dependence or abuse. The method of the present invention may be particularly useful in treating symptoms of withdrawal often observed in rehabilitation or other treatment programs. Therefore, the programs can be more effective by focusing on educational and behavioral modification goals, further reducing the incidence of program non-completion.

### EXAMPLES

### 1. Example 1

### Administration of a Benzodiazepine Compound Inhibits Cocaine Effects

Using (9aR,12aS)-4,5,6,7,9,9a,10,11,12,12a-decahydrocyclopenta[c][1,4]diazepino[6,7,1-ij]quinoline hydrochloride (Compound 1) as an example, the Rats were dosed either with vehicle or with cocaine in the presence or absence of Compound 1 (1.7 mg/kg. sc). One hour after dosing (i.p. & s.c.), rats were put in a open field, where distance moved and zones crossed were recorded by the ethovision program.

| Animal | | Number of |
|---|---|---|
| groups | Treatment | animals (n) |
| 1 | Vehicle + Vehicle | 6 |
| 2 | Vehicle + cocaine (3 mg/kg) | 6 |
| 3 | Compound 1 (1.7 mg/kg) + vehicle | 6 |
| 4 | Compound 1 (1.7 mg/kg) + cocaine (3 mg/kg) | 6 |

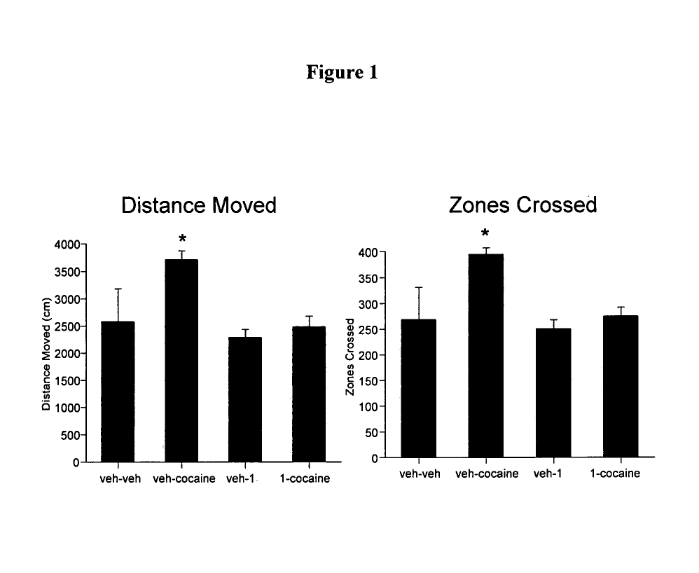

Animals were brought to the lab room 1 hour before the start of the experiment. Rats were injected with compound(s) 1 hour before being placed in the open field. Rats were then placed in open field, and the test lasted for five minutes. Results (FIG. 1) showed that cocaine (3 mg/kg, ip) produced an increase in both distance moved and the number of zones crossed. Compound 1 (1.7 mg/kg, sc) had no effect on locomotor activity alone and completely reversed the hyperactivity produced by cocaine.

### 2. Example 2

### Effect of Compounds ofFormula I on Alcohol Intake

To further illustrate the useful pharmacological properties of compounds of formula I, the effect of compound administered systematically may be determined in alcohol preferring (P) rats. Because of its pattern of drinking, the P animal seems to represent a valid genetically based model to approximate the human condition of alcoholism (McBride et al., Alcohol 7:199-205, 1990; Lankford et al., Pharmacol. Biochem. Behav. 8:293-299, 1991). After maximally preferred alcohol concentrations have stabilised for four days, test compounds are administered, for example in a dose of 2.5 and 10 mg/kg twice a day over four consecutive days. Control injections of saline are without effect on alcohol consumption in this model, whereas compounds of Formula I may reduce alcohol intake, both in terms of absolute g/kg and in proportion of alcohol to total fluid intake. Compounds of formula I may, for example, reduce intake of alcohol.

### 3. Example 3

### Additional Assays

One of ordinary skill in the art would recognize that additional assays are useful for testing the effectivenes of compounds of the present invention in methods of the present invention. Another such method is described by Pastor R. Couceyro, et al, “CART Peptides Modulate the Locomotor and Motivational Properties of Psychostimulants” JPET Fast Forward. Published on Aug. 11, 2005 as DOI:10.1124/jpet.105.091678, the entirety of which is hereby incorporated herein by reference.

The entire disclosure of each patent, patent application, and publication cited or described in this document is hereby incorporated by reference.

We claim:

1. A method for treating a patient suffering from, or susceptible to, one or more symptoms of substance abuse, dependence, or withdrawal comprising administering to said patient a compound of formula I: 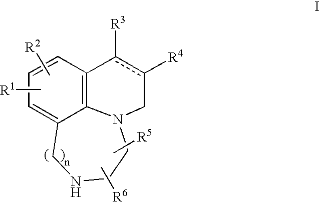
or a pharmaceutically acceptable salt thereof, wherein: [symbol]
designates a single or double bond;
n is 1 or 2;
R¹ and R² are each independently halogen, —CN, —R, —OR, —C₁₋₆ perfluoroalkyl, —OC₁₋₆ perfluoroalkyl, or phenyl optionally substituted with one to five groups independently selected from halogen, —R, —OR, —C₁₋₆ perfluoroalkyl, or —OC₁₋₆ perfluoroalkyl;
each R is independently hydrogen or a C₁₋₆ alkyl group;
R³ and R⁴ are taken together, with the carbon atoms to which they are bound, to form a saturated or unsaturated 4-8 membered carbocyclic ring, wherein said ring is optionally substituted with 1-3 groups independently selected from halogen, —R, or —OR; and
R⁵ and R⁶ are each independently —R.

2. The method according to claim 1, wherein R¹ and R² are each independently R, OR, halogen, cyano, or —C₁₋₃ perfluoroalkyl.

3. The method according to claim 2, wherein R¹ and R² are each independently hydrogen, halogen, cyano, —OR wherein R is hydrogen, C₁₋₃ alkyl, or trifluoromethyl.

4. The method according to claim 3, wherein each of R¹ and R² is hydrogen.

5. The method according to claim 1, wherein R³ and R⁴ are taken together to form a saturated or unsaturated 5-8 membered carbocyclic ring, wherein said ring is optionally substituted with 1-3 groups independently selected from halogen, —R, or OR.

6. The method according to claim 5, wherein R³ and R⁴ are taken together to form a saturated or unsaturated 5-6 membered carbocyclic ring, wherein said ring is optionally substituted with 1-3 groups independently selected from halogen, —R, or OR.

7. The method according to claim 6, wherein said compound is of formula II: 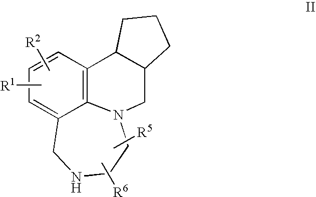
or a pharmaceutically acceptable salt thereof.

8. The method according to claim 1, wherein said compound is of formula I-a or I-b: 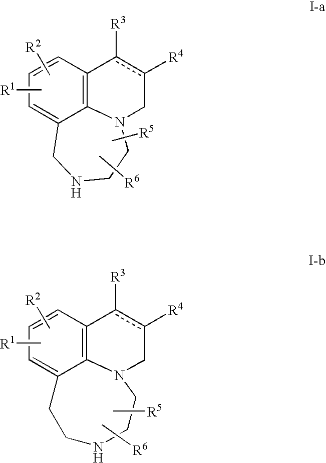
or a pharmaceutically acceptable salt thereof.

9. The method according to claim 1, wherein said compound is of formula I-c or I-d: 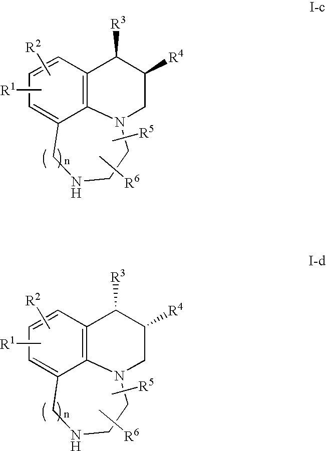
or a pharmaceutically acceptable salt thereof.

10. The method according to claim 1, wherein said compound is of formula III: 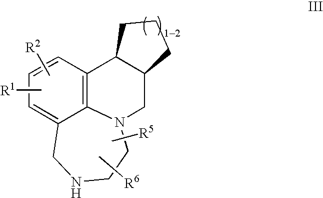
or a pharmaceutically acceptable salt thereof.

11. The method according to claim 1, wherein said compound is selected from:
4,5,6,7,9a,10,11,12,13,13a-decahydro-9H-[1,4]diazepino[6,7,1-de]phenanthridine;
2-bromo-4,5,6,7,9,9a,10,11,12,12a-decahydrocyclopenta[c][1,4]diazepino[6,7,1-ij]quinoline;
2-bromo-4,5,6,7,9,9a,10,11,12,13,14,14a-dodecahydrocyclohepta[c][1,4]diazepino[6,7,1-ij]quinoline;
2-chloro-4,5,6,7,9,9a,10,11,12,12a-decahydrocyclopenta[c][1,4]diazepino[6,7,1-ij]quinoline;
2-chloro-4,5,6,7,9,9a,10,11,12,13,14,14a-dodecahydrocyclohepta[c][1,4]diazepino[6,7,1-ij]quinoline hydrochloride;
2-phenyl-4,5,6,7,9,9a,10,11,12,12a-decahydrocyclopenta[c][1,4]diazepino[6,7,1-ij]quinoline;
2-methoxy-4,5,6,7,9,9a,10,11,12,12a-decahydrocyclopenta[c][1,4]diazepino[6,7,1-ij]quinoline;
1-fluoro-4,5,6,7,9,9a,10,11,12,12a-decahydrocyclopenta[c][1,4]diazepino[6,7,1-ij]quinoline;
1-fluoro-4,5,6,7,9,9a,10,11,12,13,14,14a-dodecahydrocyclohepta[c][1,4]diazepino[6,7,1-ij]quinoline;
1-(trifluoromethyl)-4,5,6,7,9,9a,10,11,12,12a-decahydrocyclopenta[c][1,4]diazepino[6,7,1-ij]quinoline;
1-fluoro-2-methoxy-4,5,6,7,9,9a,10,11,12,12a-decahydrocyclopenta[c][1,4]diazepino[6,7,1-ij]quinoline;
1-fluoro-2-methoxy-4,5,6,7,9,9a,10,11,12,13,14,14a-dodecahydrocyclohepta[c][1,4]diazepino[6,7,1-ij]quinoline;
4,5,6,7,9,9a10,11,12,12a-decahydrocyclopenta[c][1,4]diazepino[6,7,1-ij]quinoline;
4,5,6,7,9,9a,10,11,12,13,14,14a-dodecahydrocyclohepta[c][1,4]diazepino[6,7,1-ij]quinoline;
4,5,6,7,9,9a,10,11,12,12a-decahydrocyclopenta[c][1,4]diazepino[6,7,1-ij]quinoline;
(9aR,12aS)-4,5,6,7,9,9a,10,11,12,12a-decahydrocyclopenta[c][1,4]diazepino[6,7,1-ij]quinoline;
(9aS,12aR)-4,5,6,7,9,9a,10,11,12,12a-decahydrocyclopenta[c][1,4]diazepino[6,7,1-ij]quinoline;
(9aR,14aS)-4,5,6,7,9,9a,10,11,12,13,14,14a-dodecahydrocyclohepta[c][1,4]diazepino[6,7,1-ij]quinoline; or
(9aS,14aR)-4,5,6,7,9,9a,10,11,12,13,14,14a-dodecahydrocyclohepta[c][1,4]diazepino[6,7,1-ij]quinoline;
or a pharmaceutically acceptable salt thereof.

12. A method for treating a patient suffering from, or susceptible to, one or more symptoms of substance abuse, dependence, or withdrawal comprising administering to the patient a composition comprising a compound according to claim 1, and one or more pharmaceutically acceptable carriers.

13. The method according to claim 12, wherein said substance is a recreational substance, a pharmacologic agent, or an illicit drug.

14. The method according to claim 13, wherein said substance is a pain reliever, a tranquilizer, a stimulant, a tobacco product, or a sedative.

15. The method according to claim 13, wherein said substance is Vicodin®, Lortab®, Lorcet®, Percocet®, Percodan®, Tylox®, Hydrocodone, OxyContin®, methadone, Tramadol, marijuana, heroine, cocaine, ecstasy, LSD, PCP, or methamphetamine.
